(19) United States
(12) Patent Application Publication
(10) Pub. No.: US 2021/0090736 A1
(43) Pub. Date: Mar. 25, 2021

(54) SYSTEMS AND METHODS FOR ANOMALY DETECTION FOR A MEDICAL PROCEDURE

(71) Applicant: SHANGHAI UNITED IMAGING INTELLIGENCE CO., LTD., Shanghai (CN)

(72) Inventors: Arun Innanje, Cambridge, MA (US); Ziyan Wu, Cambridge, MA (US); Abhishek Sharma, Cambridge, MA (US); Srikrishna Karanam, Cambridge, MA (US)

(73) Assignee: SHANGHAI UNITED IMAGING INTELLIGENCE CO., LTD., Shanghai (CN)

(21) Appl. No.: 16/580,053

(22) Filed: Sep. 24, 2019

(51) Int. Cl. G16H 50/20; G06N 20/00; G16H 30/40

(52) U.S. Cl. CPC G16H 50/20; G16H 30/40; G06N 20/00

(57) ABSTRACT

The present disclosure relates to systems and methods for anomaly detection for a medical procedure. The method may include obtaining image data collected by one or more visual sensors via monitoring a medical procedure and a trained machine learning model for anomaly detection. The method may include determining a detection result for the medical procedure based on the image data using the trained machine learning model. The detection result may include whether an anomaly regarding the medical procedure exists. In response to the detection result that the anomaly exists, the method may further include providing feedback relating to the anomaly.

### TECHNICAL FIELD

The present disclosure generally relates to anomaly detection field and in particular, to systems and methods for anomaly detection for a medical procedure.

### BACKGROUND

Medical procedures (e.g., a medical scan, a surgery) in the hospital are usually sensitive to alien objects. For example, metallic objects in a magnetic resonance (MR) scanning room may cause damage to the scanner and a patient, and lead to an undesired scanning result, such as artifacts in an image generated based on the MR scan. As another example, in an operative environment, objects (e.g., sponges, needles, etc.) in a surgery procedure may be inadvertently left behind in a patient's body. Conventionally, in order to detect and/or track these objects, a magnetic tracker may be used to detect magnetically active elements in a medical scanning procedure, or the objects may be tagged with a radiofrequency identification (RFID) tag, a barcode, etc. Such trackers or objects may be susceptible to human errors. For instance, an operator (e.g., a nurse, a technician) forgets to remove a wheelchair from the MRI room or an RFID tag gets broken, etc. Therefore, it is desirable to provide a system and method to effectively and generically detect objects of interest for a medical procedure.

### SUMMARY

According to an aspect of the present disclosure, a system for anomaly detection for a medical procedure is provided. The system may include at least one storage device storing executable instructions and at least one processor in communication with the at least one storage device. When executing the executable instructions, the at least one processor may cause the system to perform the following operations. The system may obtain image data collected by one or more visual sensors via monitoring a medical procedure. The system may obtain a trained machine learning model for anomaly detection. The system may determine a detection result for the medical procedure based on the image data using the trained machine learning model. The detection result may include whether an anomaly regarding the medical procedure exists. In response to the detection result that the anomaly regarding the medical procedure exists, the system may determine location information of at least one of one or more objects of interest based on the image data using the trained machine learning model. In response to the detection result that the anomaly exists, the system may provide feedback relating to the anomaly.

In some embodiments, to provide feedback relating to the anomaly, the system may generate a notification for notifying that the anomaly exists.

In some embodiments, the image data may include representation of one or more objects of interest that cause the anomaly.

In some embodiments, to determine the location information of at least one of the one or more objects of interest, the system may extract a plurality of regions represented in the image data. The system may determine a score of each of the plurality of regions. The score of each of the plurality of regions may denote a probability that the each of the plurality of regions includes the at least one of the one or more objects of interest. The system may also determine the location information of the at least one of the one or more objects of interest in the image data based on the score of each of the plurality of regions.

In some embodiments, to provide feedback relating to the anomaly, the system may cause at least a portion of the image data to be presented as a presentation on a device. The system may cause the at least one of the one or more objects to be highlighted in the presentation.

In some embodiments, the presentation may be in a form of a video or a static image.

In some embodiments, the trained machine learning model for anomaly detection may be constructed based on a weakly supervised learning model.

In some embodiments, the trained machine learning model may be provided by the following operations. The system may obtain a plurality of training samples. Each of the plurality of training samples may include a label indicating whether a training sample includes a sample anomaly. The system may determine a plurality of regions in each of the plurality of training samples. Each of at least a portion of the plurality of regions may include an object. The system may extract image features from each of the plurality of regions. The system may train an initial machine learning model using the extracted image features and the labels of the plurality of training samples.

In some embodiments, the plurality of training samples may include a plurality of negative training samples each of which has no sample anomaly.

In some embodiments, the plurality of training samples may include a first portion and a second portion. The first portion may include a plurality of negative training samples. Each of the plurality of negative training samples may have no sample anomaly. The second portion may include a plurality of positive training samples. Each of the plurality of positive training samples may include a sample anomaly.

In some embodiments, the trained machine learning model may be constructed based on a neural network model.

According to another aspect of the present disclosure, a method may be provided. The method may be implemented on a computing device having at least one processor and at least one storage device for anomaly detection for a medical procedure. The method may include obtaining image data collected by one or more visual sensors via monitoring a medical procedure. The method may include obtaining a trained machine learning model for anomaly detection. The method may further include determining a detection result for the medical procedure based on the image data using the trained machine learning model. The detection result may include whether an anomaly regarding the medical procedure exists. In response to the detection result that the anomaly regarding the medical procedure exists, the method may include determining the location information of at least one of the one or more objects of interest based on the image data using the trained machine learning model. In response to the detection result that the anomaly exists, the method may include providing feedback relating to the anomaly.

In some embodiments, to provide feedback relating to the anomaly, the method may further include generating a notification for notifying that the anomaly exists.

In some embodiments, to determine the location information of at least one of the one or more objects of interest, the method may further include the following operations. The method may include extracting a plurality of regions represented in the image data. The method may include determining a score of each of the plurality of regions. The score of each of the plurality of regions may denote a probability that the each of the plurality of regions includes the at least one of the one or more objects of interest. The method may include determining the location information of the at least one of the one or more objects of interest in the image data based on the score of each of the plurality of regions.

In some embodiments, to provide feedback relating to the anomaly, the method may include causing at least a portion of the image data to be presented as a presentation on a device. The method may include causing the at least one of the one or more objects to be highlighted in the presentation.

In some embodiments, the trained machine learning model may be provided by the following operations. The method may include obtaining a plurality of training samples, each of which may include a label indicating whether a training sample includes a sample anomaly. The method may include determining a plurality of regions in each of the plurality of training samples, and each of at least a portion of the plurality of regions may include an object. The method may include extracting image features from each of the plurality of regions. The method may further include training an initial machine learning model using the extracted image features and the labels of the plurality of training samples.

According to yet another aspect of the present disclosure, a non-transitory computer readable medium is provided. The non-transitory computer readable medium may include a set of instructions for anomaly detection for a medical procedure. When executed by at least one processor, the set of instructions may direct the at least one processor to effectuate a method. The method may include obtaining image data collected by one or more visual sensors via monitoring a medical procedure. The method may include obtaining a trained machine learning model for anomaly detection. The method may include determining a detection result for the medical procedure based on the image data using the trained machine learning model. The detection result may include whether an anomaly regarding the medical procedure exists. In response to the detection result that the anomaly regarding the medical procedure exists, the method may include determining the location information of at least one of the one or more objects of interest based on the image data using the trained machine learning model. In response to the detection result that the anomaly exists, the method may further include providing feedback relating to the anomaly.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary anomaly detection system according to some embodiments of the present disclosure;

FIG. 2 is a schematic diagram illustrating hardware and/or software components of an exemplary computing device according to some embodiments of the present disclosure;

FIG. 3 is a schematic diagram illustrating hardware and/or software components of a mobile device according to some embodiments of the present disclosure;

FIG. 4A is a block diagram illustrating an exemplary processing device according to some embodiments of the present disclosure;

FIG. 4B is a block diagram illustrating another exemplary processing device according to some embodiments of the present disclosure;

FIG. 5 is a flowchart illustrating an exemplary process of anomaly detection according to some embodiments of the present disclosure;

FIG. 6 is a flowchart illustrating an exemplary process of training a machine learning model according to some embodiments of the present disclosure;

FIG. 7 is a schematic diagram illustrating a detection result regarding an exemplary medical procedure according to some embodiments of the present disclosure;

FIG. 8 is a schematic diagram illustrating a detection result regarding another exemplary medical procedure according to some embodiments of the present disclosure; and

FIG. 9 is a schematic diagram illustrating an anomaly detection of an exemplary surgery procedure according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprises,” “comprising,” “includes,” and/or “including” when used in this disclosure, specify the presence of stated features, integers, steps, operations, elements, and/or components, but do not preclude the presence or addition of one or more other features, integers, steps, operations, elements, components, and/or groups thereof.

These and other features, and characteristics of the present disclosure, as well as the methods of operations and functions of the related elements of structure and the combination of parts and economies of manufacture, may become more apparent upon consideration of the following description with reference to the accompanying drawing(s), all of which form part of this specification. It is to be expressly understood, however, that the drawing(s) is for the purpose of illustration and description only and are not intended to limit the scope of the present disclosure. It is understood that the drawings are not to scale.

The flowcharts used in the present disclosure illustrate operations that systems implement according to some embodiments of the present disclosure. It is to be expressly understood, the operations of the flowcharts may be implemented not in order. Conversely, the operations may be implemented in inverted order, or simultaneously. Moreover, one or more other operations may be added to the flowcharts. One or more operations may be removed from the flowcharts.

An aspect of the present disclosure relates to methods and systems for anomaly detection for a medical procedure. The system may obtain image data collected by monitoring a medical procedure using one or more visual sensors. The system may obtain a trained machine learning model for anomaly detection. The system may also determine a detection result for the medical procedure using the trained machine learning model. The detection result may include whether an anomaly regarding the medical procedure exists based on the image data. In response to the detection result that the anomaly exists, the system may provide feedback relating to the anomaly. In this way, the anomaly detection system may detect whether the anomaly regarding medical procedure exists effectively and generically. As used herein, the term “generically” indicates that the anomaly detection system may be applied to detect anomaly due to various alien objects that may cause damage or abnormity to the medical device, an individual, etc., associated with the medical procedure, and not specific to a specific type of alien objects. The anomaly detection system may further determine location information of one or more objects that cause the anomaly regarding the medical procedure and provide feedback. The methods and systems for anomaly detection according to some embodiments of the present disclosure may reduce the risk of anomaly due to the existence of one or more alien objects in a medical procedure to individuals, medical devices, etc., associated with the medical procedure. Accordingly, the systems and methods as described herein may perform an automated anomaly detection based on image processing. For example, the systems and methods may input an image regarding a medical procedure into a trained machine learning model. The trained machine learning model may directly and automatically output a detection result including whether an anomaly regarding the medical procedure exists via processing the image. The systems and methods as described herein may identify in real time an anomaly regarding a medical procedure and objects causing the anomaly although the objects causing the anomaly are various.

It should be noted that the anomaly detection system 100 described below is merely provided for illustration purposes, and not intended to limit the scope of the present disclosure. For persons having ordinary skills in the art, a certain amount of variations, changes, and/or modifications may be deducted under the guidance of the present disclosure. Those variations, changes, and/or modifications do not depart from the scope of the present disclosure.

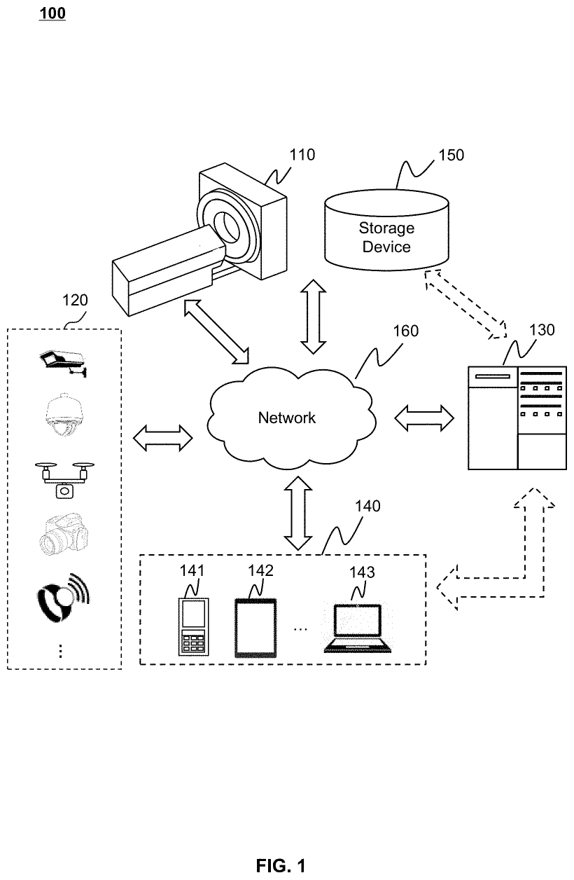

FIG. 1 is a schematic diagram illustrating an exemplary anomaly detection system 100 according to some embodiments of the present disclosure. In some embodiments, the anomaly detection system 100 may be used in an intelligent transportation system (ITS), a security system, a transportation management system, a prison system, an astronomical observation system, a monitoring system, a species identification system, an industry controlling system, an identity identification (ID) system, a medical procedure system, a retrieval system, or the like, or any combination thereof. The anomaly detection system 100 may be a platform for data and/or information processing, for example, training a machine learning model for anomaly detection and/or data classification, such as image classification, text classification, etc. The anomaly detection system 100 may be applied in intrusion detection, fault detection, network abnormal traffic detection, fraud detection, behavior abnormal detection, or the like, or a combination thereof. An anomaly may be also referred to as an outlier, a novelty, a noise, a deviation, an exception, etc. As used herein, an anomaly refers to an action or an event that is determined to be unusual or abnormal in view of known or inferred conditions. For example, for an examination procedure in a police office, prison, etc., the anomaly may include anomaly due to the existence of an alien object. As another example, for a medical procedure, the anomaly may include anomaly caused by an individual's behavior, anomaly due to the existence of an alien object, etc.

For the purposes of illustration, the anomaly detection system 100 used in as a medical procedure system may be described. As illustrated in FIG. 1, the anomaly detection system 100 may include a medical device 110, a monitoring device 120, one or more terminal(s) 140, a processing device 130, a storage device 150, and a network 160. In some embodiments, the medical device 110, the monitoring device 120, the processing device 130, the terminal(s) 140, and/or the storage device 150 may be connected to and/or communicate with each other via a wireless connection (e.g., the network 160), a wired connection, or a combination thereof. The connections between the components in the anomaly detection system 100 may vary. Merely by way of example, the monitoring device 120 may be connected to the processing device 130 through the network 160, as illustrated in FIG. 1. As another example, the storage device 150 may be connected to the processing device 130 through the network 160, as illustrated in FIG. 1, or connected to the processing device 130 directly. As a further example, the terminal(s) 140 may be connected to the processing device 130 through the network 160, as illustrated in FIG. 1, or connected to the processing device 130 directly.

The medical device 110 may include any device used in a medical procedure. As used herein, a medical procedure may refer to an activity or a series of actions attended to achieve a result in the delivery of healthcare, for example, directed at or performed on a subject (e.g., a patient) to measure, diagnosis and/or treat the subject. Exemplary medical procedures may include an immediate test, a diagnostic test, a treatment procedure, an autopsy, etc. The immediate test may be performed before an initial illness or condition is addressed to check the overall health condition of an individual. A result of the immediate test may be obtained in real-time when the immediate test is performed. For example, the immediate test may include a blood pressure test. A diagnostic test may be performed to check for certain conditions or diseases or to test the body's endurance. For example, the diagnostic test may include a cardio stress test used to test the strength of the heart, an imaging scan for a partial or the entire body of a patient, a surgery for diagnosis, etc. A treatment procedure may include a series of actions to correct a problem or a disease of a subject (e.g., a patient). For example, a treatment procedure may include surgery, radiotherapy, etc. The subject may be biological or non-biological. For example, the subject may include a patient, a man-made object, etc. As another example, the subject may include a specific portion, organ, and/or tissue of the patient. For example, the subject may include head, neck, thorax, heart, stomach, blood vessel, soft tissue, tumor, nodules, or the like, or a combination thereof.

The medical device 110 may include an imaging device, a treatment device (e.g., surgical equipment), a multi-modality device to acquire one or more images of different modalities or acquire an image relating to at least one part of a subject and perform treatment on the at least one part of the subject, etc. The imaging device may be configured to generate an image including a representation of at least one part of the subject. Exemplary imaging devices may include, for example, a computed tomography (CT) device, a cone beam CT device, a positron emission computed tomography (PET) device, a volume CT device, a magnetic resonance imaging (MRI) device, or the like, or a combination thereof. The treatment device may be configured to perform a treatment on at least one part of the subject. Exemplary treatment devices may include a radiotherapy device (e.g., a linear accelerator), an X-ray treatment device, surgery equipment, etc. Exemplary surgical equipment may include an anesthesia machine, a respirator, an operation table, a lamp, an infusion pump, surgical consumables (e.g., a tourniquet, sponges, etc.), or any other instruments, such as scalpels, hemostatic forceps, etc.

The monitoring device 120 may be positioned to perform surveillance of an area of interest (AOI) or an object of interest within the scope of the monitoring device 120. The monitoring device 120 may include one or more acoustic sensors, one or more visual sensors, etc. The one or more acoustic sensors may be configured to collect audio signals and/or generate audio data from a medical procedure. For example, the one or more acoustic sensors may be a microphone, a recorder, etc., which may collect audio signals when an individual (e.g., a doctor, a patient, etc.) speaks and convert the collected audio signals into digital signals (i.e., audio data). The visual sensors may refer to an apparatus for visual recording. The visual sensors may capture image data for recording a medical procedure. The image data may include a static image, a video, an image sequence including multiple static images, etc. In some embodiments, the visual sensors may include a stereo camera configured to capture a static image or video. The stereo camera may include a binocular vision device or a multi-camera. In some embodiments, the visual sensors may include a digital camera. The digital camera may include a 2D camera, a 3D camera, a panoramic camera, a VR (virtual reality) camera, a web camera, an instant picture camera, an IR camera, an RGB-D camera, or the like, or any combination thereof. The digital camera may be added to or be part of a medical imaging equipment, a night vision equipment, a radar system, a sonar system, an electronic eye, a camcorder, a thermal imaging device, a smartphone, a tablet PC, a laptop, a wearable device (e.g., 3D glasses), an eye of a robot, or the like, or any combination thereof. The digital camera may also include an optical sensor, a radio detector, an artificial retina, a mirror, a telescope, a microscope, or the like, or any combination thereof. In some embodiments, the monitoring device 120 may transmit the collected image data and/or audio data to the processing device 130, the storage device 150 and/or the terminal(s) 140 via the network 160.

The processing device 130 may process data and/or information obtained from the medical device 110, the monitoring device 120, the terminal(s) 140, the storage device 150, and/or the monitoring device 120. For example, the processing device 130 may process image data captured by the monitoring device 120. As another example, the processing device may train a machine learning model to obtain a trained machine learning model for anomaly detection. As still another example, the processing device 130 may determine a detection result for a medical procedure based on the image data using the trained machine learning model for anomaly detection. In some embodiments, the determination and/or updating of the trained machine learning model may be performed on a processing device, while the application of the trained machine learning model may be performed on a different processing device. In some embodiments, the determination and/or updating of the trained machine learning model may be performed on a processing device of a system different than the anomaly detection system 100 or a server different than a server including the processing device 130 on which the application of the trained machine learning model is performed. For instance, the determination and/or updating of the trained machine learning model may be performed on a first system of a vendor who provides and/or maintains such a machine learning model and/or has access to training samples used to determine and/or update the trained machine learning model, while anomaly detection of a medical procedure based on the provided machine learning model, may be performed on a second system of a client of the vendor. In some embodiments, the determination and/or updating of the trained machine learning model may be performed online in response to a request for anomaly detection of a medical procedure. In some embodiments, the determination and/or updating of the trained machine learning model may be performed offline.

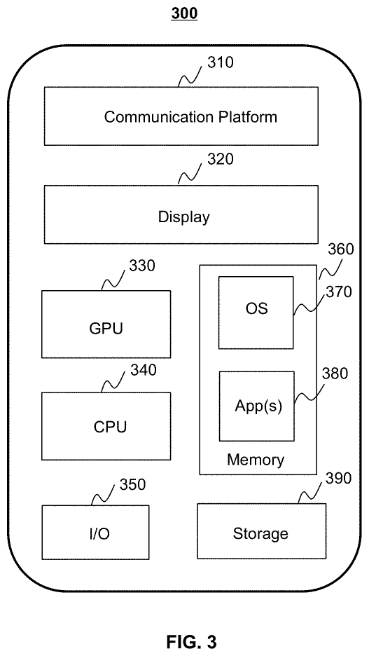

In some embodiments, the processing device 130 may be a single server or a server group. The server group may be centralized or distributed. In some embodiments, the processing device 130 may be local or remote. For example, the processing device 130 may access information and/or data from the medical device 110, the terminal(s) 140, the storage device 150, and/or the monitoring device 120 via the network 160. As another example, the processing device 130 may be directly connected to the medical device 110, the monitoring device 120, the terminal(s) 140, and/or the storage device 150 to access information and/or data. In some embodiments, the processing device 130 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 130 may be implemented by a mobile device 300 having one or more components as described in connection with FIG. 3.

The terminal(s) 140 may be connected to and/or communicate with the medical device 110, the processing device 130, the storage device 150, and/or the monitoring device 120. For example, the terminal(s) 140 may obtain a processed image from the processing device 130. As another example, the terminal(s) 140 may obtain image data acquired via the monitoring device 120 and transmit the image data to the processing device 130 to be processed. In some embodiments, the terminal(s) 140 may include a mobile device 141, a tablet computer 142, . . . , a laptop computer 143, or the like, or any combination thereof. For example, the mobile device 141 may include a mobile phone, a personal digital assistance (PDA), a gaming device, a navigation device, a point of sale (POS) device, a laptop, a tablet computer, a desktop, or the like, or any combination thereof. In some embodiments, the terminal(s) 140 may include an input device, an output device, etc. The input device may include alphanumeric and other keys that may be input via a keyboard, a touch screen (for example, with haptics or tactile feedback), a speech input, an eye-tracking input, a brain monitoring system, or any other comparable input mechanism. The input information received through the input device may be transmitted to the processing device 130 via, for example, a bus, for further processing. Other types of the input device may include a cursor control device, such as a mouse, a trackball, or cursor direction keys, etc. The output device may include a display, a speaker, a printer, or the like, or a combination thereof. In some embodiments, the terminal(s) 140 may be part of the processing device 130.

The storage device 150 may store data, instructions, a machine learning model (e.g., an initial machine learning model, a trained machine learning model, etc.), and/or any other information. In some embodiments, the storage device 150 may store data obtained from the medical device 110, the terminal(s) 140, the processing device 130, and/or the monitoring device 120. In some embodiments, the storage device 150 may store data and/or instructions that the processing device 130 may execute or use to perform exemplary methods described in the present disclosure. In some embodiments, the storage device 150 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 150 may be implemented on a cloud platform as described elsewhere in the disclosure.

In some embodiments, the storage device 150 may be connected to the network 160 to communicate with one or more other components in the anomaly detection system 100 (e.g., the processing device 130, the terminal(s) 140, the visual sensor, etc.). One or more components in the anomaly detection system 100 may access the data or instructions stored in the storage device 150 via the network 160. In some embodiments, the storage device 150 may be part of the processing device 130.

The network 160 may include any suitable network that can facilitate the exchange of information and/or data for the anomaly detection system 100. In some embodiments, one or more components of the anomaly detection system 100 (e.g., the medical device 110, the terminal(s) 140, the processing device 130, the storage device 150, the monitoring device 120, etc.) may communicate information and/or data with one or more other components of the anomaly detection system 100 via the network 160. For example, the processing device 130 may obtain image data from the visual sensor via the network 160. As another example, the processing device 130 may obtain user instruction(s) from the terminal(s) 140 via the network 160. The network 160 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (VPN), a satellite network, a telephone network, routers, hubs, switches, server computers, and/or any combination thereof. For example, the network 160 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 160 may include one or more network access points. For example, the network 160 may include wired and/or wireless network access points such as base stations and/or internet exchange points through which one or more components of the anomaly detection system 100 may be connected to the network 160 to exchange data and/or information.

This description is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. For example, the storage device 150 may be a data storage including cloud computing platforms, such as public cloud, private cloud, community, and hybrid clouds, etc. However, those variations and modifications do not depart the scope of the present disclosure.

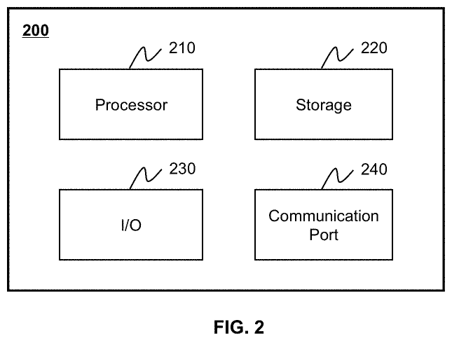

FIG. 2 is a schematic diagram illustrating hardware and/or software components of an exemplary computing device according to some embodiments of the present disclosure. As illustrated in FIG. 2, the computing device 200 may include a processor 210, a storage 220, an input/output (I/O) 230, and a communication port 240. In some embodiments, the processing device 130 and/or the terminal(s) 140 may be implemented on the computing device 200.

The processor 210 may execute computer instructions (program code) and, when executing the instructions, cause the processing device 130 to perform functions of the processing device 130 in accordance with techniques described herein. The computer instructions may include, for example, routines, programs, objects, components, signals, data structures, procedures, modules, and functions, which perform particular functions described herein. In some embodiments, the processor 210 may process data and/or images obtained from the medical device 110, the terminal(s) 140, the storage device 150, the monitoring device 120, and/or any other component of the anomaly detection system 100. For example, the processor 210 may obtain an image from the monitoring device 120 and process the image to obtain features of the image. In some embodiments, the processor 210 may include one or more hardware processors, such as a microcontroller, a microprocessor, a reduced instruction set computer (RISC), an application-specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field-programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both process A and process B, it should be understood that process A and process B may also be performed by two or more different processors jointly or separately in the computing device 200 (e.g., a first processor executes process A and a second processor executes process B, or the first and second processors jointly execute processes A and B).

The storage 220 may store data/information obtained from the medical device 110, the terminal(s) 140, the storage device 150, the monitoring device 120, or any other component of the anomaly detection system 100. In some embodiments, the storage 220 may include a mass storage device, removable storage device, a volatile read-and-write memory, a read-only memory (ROM), or the like, or any combination thereof. For example, the mass storage may include a magnetic disk, an optical disk, a solid-state drive, etc. The removable storage may include a flash drive, a floppy disk, an optical disk, a memory card, a zip disk, a magnetic tape, etc. The volatile read-and-write memory may include a random access memory (RAM). The RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. The ROM may include a mask ROM (MROM), a programmable ROM (PROM), an erasable programmable ROM (PEROM), an electrically erasable programmable ROM (EEPROM), a compact disk ROM (CD-ROM), and a digital versatile disk ROM, etc. In some embodiments, the storage 220 may store one or more programs and/or instructions to perform exemplary methods described in the present disclosure. For example, the storage 220 may store a program (e.g., in the form of computer-executable instructions) for the processing device 130 for training an initial machine learning model to generate a trained machine learning model. As another example, the storage 220 may store a program (e.g., in the form of computer-executable instructions) for the processing device 130 for detecting one or more objects in image data using the trained machine learning model.

The I/O 230 may input or output signals, data, and/or information. In some embodiments, the I/O 230 may enable user interaction with the processing device 130. In some embodiments, the I/O 230 may include an input device and an output device. Exemplary input devices may include a keyboard, a mouse, a touch screen, a microphone, or the like, or a combination thereof. Exemplary output devices may include a display device, a loudspeaker, a printer, a projector, or the like, or a combination thereof. Exemplary display devices may include a liquid crystal display (LCD), a light-emitting diode (LED)-based display, a flat panel display, a curved screen, a television device, a cathode ray tube (CRT), or the like, or a combination thereof.

The communication port 240 may be connected to a network (e.g., the network 160) to facilitate data communications. The communication port 240 may establish connections between the processing device 130 and the medical device 110, the terminal(s) 140, the storage device 150, or the monitoring device 120. The connection may be a wired connection, a wireless connection, or a combination of both that enables data transmission and reception. The wired connection may include an electrical cable, an optical cable, a telephone wire, or the like, or any combination thereof. The wireless connection may include Bluetooth, Wi-Fi, WiMAX, WLAN, ZigBee, mobile network (e.g., 3G, 4G, 5G, etc.), or the like, or a combination thereof. In some embodiments, the communication port 240 may be a standardized communication port, such as RS232, RS485, etc. In some embodiments, the communication port 240 may be a specially designed communication port. For example, the communication port 240 may be designed in accordance with the digital imaging and communications in medicine (DICOM) protocol.

FIG. 3 is a schematic diagram illustrating hardware and/or software components of a mobile device according to some embodiments of the present disclosure. In some embodiments, the processing device 130 and/or the terminal(s) 140 may be implemented on the computing device 200. As illustrated in FIG. 3, the mobile device 300 may include a communication platform 310, a display 320, a graphics processing unit (GPU) 330, a central processing unit (CPU) 340, an I/O 350, a memory 360, and storage 390. In some embodiments, any other suitable component, including but not limited to a system bus or a controller (not shown), may also be included in the mobile device 300. In some embodiments, a mobile operating system 370 (e.g., iOS, Android, Windows Phone, etc.) and one or more applications 380 may be loaded into the memory 360 from the storage 390 in order to be executed by the CPU 340. The applications 380 may include a browser or any other suitable mobile apps for receiving and rendering information relating to image processing or other information from the processing device 130. User interactions with the information stream may be achieved via the I/O 350 and provided to the processing device 130 and/or other components of the anomaly detection system 100 via the network 160.

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar therewith to adapt those technologies to generate a high-quality image of a scanned object as described herein. A computer with user interface elements may be used to implement a personal computer (PC) or another type of work station or terminal device, although a computer may also act as a server if appropriately programmed. It is believed that those skilled in the art are familiar with the structure, programming and general operation of such computer equipment and as a result, the drawings should be self-explanatory.

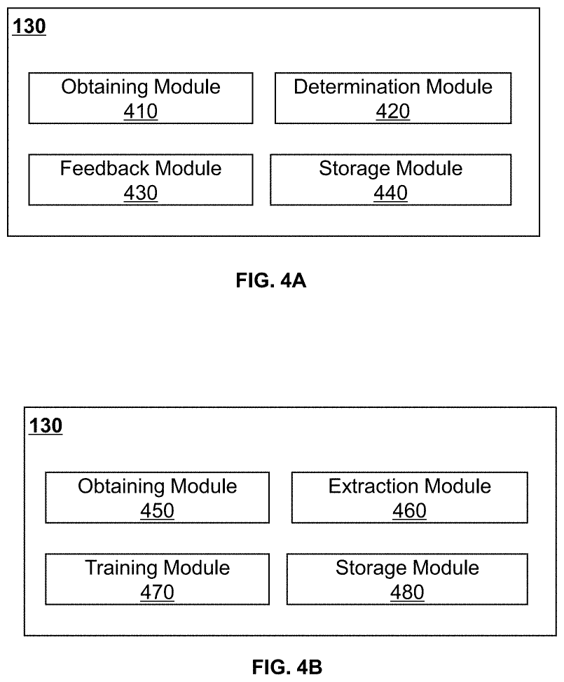

FIG. 4A is a block diagram illustrating an exemplary processing device according to some embodiments of the present disclosure. In some embodiments, the processing device 130 may be implemented on a computing device 200 (e.g., the processor 210) illustrated in FIG. 2 or a CPU 340 as illustrated in FIG. 3. As illustrated in FIG. 4A, the processing device 130 may include an obtaining module 410, a determination module 420, a feedback module 430, and a storage module 440. Each of the modules described above may be a hardware circuit that is designed to perform certain actions, e.g., according to a set of instructions stored in one or more storage media, and/or any combination of the hardware circuit and the one or more storage media.

The obtaining module 410 may be configured to obtain information and/or data for anomaly detection for a medical procedure. For example, the obtaining module 410 may obtain image data collected by one or more visual sensors via monitoring a medical procedure. As another example, the obtaining module 410 may obtain a trained machine learning model for anomaly detection. The trained machine learning model for anomaly detection may be configured to detect an anomaly regarding a specific medical procedure based on specific image data associated with the specific medical procedure. The trained machine learning model may be used to identify and/or determine location information of one or more objects of interest in the inputted specific image data that cause the anomaly regarding the specific medical procedure in response to the determination that the anomaly regarding the specific medical procedure exists. In some embodiments, the obtaining module 410 may obtain the image data or the trained machine learning model from the monitoring device 120, the storage device 150, the terminal(s) 140, or any other storage device from time to time, e.g., periodically or in real-time. For example, the image data may be collected by the monitoring device 120 and transmitted to the one or more components of the anomaly detection system 100.

The determination module 420 may determine a detection result for the medical procedure using the trained machine learning model based on the image data. The determination module 420 may input the image data into the trained machine learning model. The determination module 420 may obtain the detection result generated using the trained machine learning model based on the inputted image data. In some embodiments, the detection result for the medical procedure may include a positive result or a negative result. The positive result may indicate the existence of the anomaly regarding the medical procedure. In some embodiments, in response to a determination that the image data includes the anomaly, the determination module 420 may identify and/or determine location information of one or more objects of interest in the image data that cause the anomaly regarding the medical procedure in response to the determination that the anomaly regarding the medical procedure exists based on the image data using the trained machine learning model.

The feedback module 430 may be configured to provide feedback relating to the anomaly in response to the detection result that the anomaly exists. In some embodiments, the feedback provided by the feedback module 430 may include the detection result that the anomaly regarding the medical procedure exists. For example, the feedback module 430 may generate a notification for notifying that the anomaly exists. The notification for notifying that the anomaly exists may be transmitted to a device (e.g., the terminal(s) 140). The device (e.g., the terminal(s) 140) may be caused to play and/or display the notification to related personal (e.g., a patient, a doctor) for notifying that the anomaly exists.

The storage module 412 may store information. The information may include programs, software, algorithms, data, text, number, images and some other information. For example, the information may include image data associated with a medical procedure, a trained machine learning model for anomaly detection, etc.

It should be noted that the above description of the processing device 130 is merely provided for the purposes of illustration, and not intended to limit the scope of the present disclosure. For persons having ordinary skills in the art, multiple variations and modifications may be made under the teachings of the present disclosure. For instance, the assembly and/or function of the processing device 130 may be varied or changed according to specific implementation scenarios. Merely by way of example, the determination module 420 and the feedback module 430 may be integrated into a single module.

FIG. 4B is a block diagram illustrating another exemplary processing device according to some embodiments of the present disclosure. In some embodiments, the processing device 130 may be implemented on a computing device 200 (e.g., the processor 210) illustrated in FIG. 2 or a CPU 340 as illustrated in FIG. 3. As illustrated in FIG. 4A, the processing device 130 may include an obtaining module 450, an extraction module 460, a training module 470, and a storage module 480. Each of the modules described above may be a hardware circuit that is designed to perform certain actions, e.g., according to a set of instructions stored in one or more storage media, and/or any combination of the hardware circuit and the one or more storage media.

The obtaining module 410 may be configured to obtain a plurality of training samples each of which includes image data (e.g., an image, a video, etc.) associated with a normal scene regarding a medical procedure associated with the training sample. In some embodiments, the obtaining module 410 may be configured to obtain a plurality of training samples a portion of which includes image data (e.g., images, videos, etc.) associated with abnormal scene regarding medical procedures. As used herein, a training sample regarding an abnormal scene may be also referred to as a sample including a sample abnormal. If a training sample includes a sample anomaly, the training sample may including a label indicating the sample anomaly. Each of the plurality of training samples may include historical image data collected by one or more visual sensors via monitoring a historical medical procedure in a historical time period (e.g., the past one or more years, the past one or more months). For example, a training sample may include one or more static images captured by the one or more visual sensors. In some embodiments, the training samples may be obtained from the monitoring device 120 or acquired from a storage device (e.g., the storage device 150, an external data source), the terminal(s) 140, or any other storage device.

In some embodiments, the label of each of the plurality of training samples may be negative label. A training sample may be tagged with a negative label if the training sample is a negative training sample with no sample anomaly. In some embodiments, each of a portion of the plurality of training samples may be tagged with a positive label if the training sample is a positive training sample with sample anomaly. A training sample may be tagged with a binary label (e.g., 0 or 1, positive or negative, etc.). For example, a negative training sample may be tagged with a negative label (e.g., “0”), while a positive training sample may be tagged with a positive label (e.g., “1”). Availability of positive samples in the plurality of training samples may increase the accuracy of a trained machine learning model for anomaly detection that is trained using the plurality of training samples.

The extraction module 460 may be configured to determine a plurality of regions in each of the plurality of training samples using an initial machine learning model. In some embodiments, the plurality of regions may be determined using the initial machine learning model based on a sliding window algorithm, a region proposal algorithm, an image segmentation algorithm, etc. In some embodiments, the extraction module 460 may be also configured to extract image features from each of the plurality of regions. An image feature may refer to a representation of a specific structure in a region of a training sample, such as a point, an edge, an object, etc. The extracted image features may be binary, numerical, categorical, ordinal, binomial, interval, text-based, or combinations thereof. In some embodiments, an image feature may include a low-level feature (e.g., an edge feature, a textural feature), a high-level feature (e.g., a semantic feature), or a complicated feature (e.g., a deep hierarchical feature). The initial machine learning model may process the inputted training sample via multiple layers of feature extraction (e.g., convolution layers) to extract image features.

The training module 470 may be configured to train the initial machine learning model to obtain a trained machine learning model. In some embodiments, the trained machine learning model may be obtained by training the initial machine learning model based on the extracted image features of each of the plurality of training samples using a training algorithm. Exemplary training algorithms may include a gradient descent algorithm, a Newton's algorithm, a Quasi-Newton algorithm, a Levenberg-Marquardt algorithm, a conjugate gradient algorithm, or the like, or a combination thereof.

The storage module 480 may store information. The information may include programs, software, algorithms, data, text, number, images and some other information. For example, the information may include training samples, a trained machine learning model for anomaly detection, an initial training machine learning model, a training algorithm, etc.

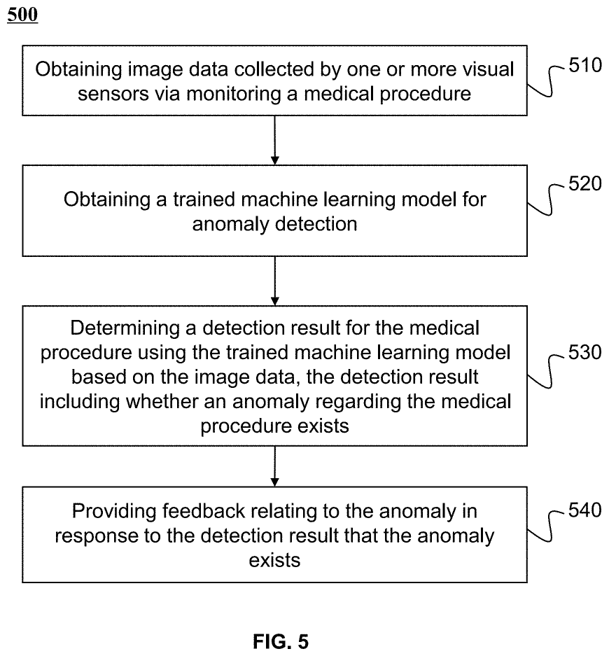

FIG. 5 is a flowchart illustrating an exemplary process 500 of anomaly detection according to some embodiments of the present disclosure. The process 500 may be executed by the anomaly detection system 100. For example, process 500 may be implemented as a set of instructions (e.g., an application) stored in the storage device 150 in the anomaly detection system 100. The processing device 130 may execute the set of instructions and may accordingly be directed to perform the process 500 in the anomaly detection system 100. The operations of the illustrated process 500 presented below are intended to be illustrative. In some embodiments, the process 500 may be accomplished with one or more additional operations not described, and/or without one or more of the operations discussed. Additionally, the order in which the operations of the process 500 as illustrated in FIG. 5 and described below is not intended to be limiting.

In 510, the processing device 130 (e.g., the obtaining module 410) may obtain image data collected by one or more visual sensors via monitoring a medical procedure.

The image data may include a representation of a scene regarding the medical procedure. For example, the image data may include a representation of one or more objects appears in the scene regarding the medical procedure. The medical procedure may include a diagnostic procedure, a treatment procedure, as described elsewhere in the present disclosure (FIG. 1 and the descriptions thereof). Merely by way of an example, the medical procedure may include an imaging scan using an imaging device (e.g., a single-photon emission tomography (SPET), an MR scanner, etc.), a surgery procedure (e.g., a cardiac surgical procedure), etc. The one or more visual sensors may be configured to perform surveillance of an area of interest (AOI) or one or more objects within the scope of the one or more visual sensors where the medical procedure is performed. More descriptions for the medical procedure and/or the visual sensors may be found in FIG. 1 and the descriptions thereof. The image data collected by the one or more visual sensors may include representations of one or more objects appear where the medical procedure is performed. An object appear where the medical procedure is performed may include an individual (e.g., a doctor, a patient), a medical device, or any other physical subject, such as an accessory of the individual (e.g., a bracelet, a necklace, or glasses), a wheelchair for a patient, etc. In some embodiments, the image data may include one or more static images, a video, or a combination thereof, acquired by the one or more visual sensors. For example, the one or more visual sensors may include an infrared (IR)camera configured to collect IR images for recording one or more scenes in the medical procedure, a video camera configured to capture a video that records the medical procedure, an RGB-D camera configured to take images that records one or more scenes in the medical procedure, etc.

In some embodiments, the image data may include a video of multiple frames. Each of the multiple frames may have a timestamp recording the time at which the one or more visual sensors capture the frame. In some embodiments, the image data may include one or more static images that may form an image sequence. Each of the one or more static images may have a timestamp recording the time at which the one or more visual sensors take the static image. The image data may record the medical procedure over the course of time according to timestamps associated with the image data. For example, the change of locations of an object (e.g., a sponge) in a surgery procedure over a period of time may be recorded by the image data based on the timestamps associated with the image data.

In some embodiments, the processing device 130 may obtain the image data from the monitoring device 120, the storage device 150, the terminal(s) 140, or any other storage device from time to time, e.g., periodically or in real-time. For example, the image data may be collected by the monitoring device 120 and transmitted to the one or more components of the anomaly detection system 100. For example, the image data collected by the monitoring device 120 may be transmitted to the processing device 130 directly in real-time for further processing. As another example, the image data collected by the monitoring device 120 may be transmitted to the storage device 150 or an external source for storage. The processing device 130 may retrieve at least a portion of the image data from the storage device 150 or an external storage device. As a further example, the image data acquired by the one or more visual sensors may be transmitted to the terminal(s) 140 for display. The processing device 130 may transmit at least a portion of the image data (e.g., after processing) to the terminal(s) 140 via the network 160.

In 520, the processing device 130 (e.g., the obtaining module 410) may obtain a trained machine learning model for anomaly detection. In some embodiments, the trained machine learning model for anomaly detection may be configured to detect an anomaly regarding a specific medical procedure based on specific image data associated with the specific medical procedure. As used herein, an anomaly in a specific medical procedure may refer to occurrence or existence of one or more objects of interest in the medical procedure, which may cause damage or abnormity to the medical device (e.g., the medical device 110), an individual (e.g., a doctor, the patient, etc.), etc., associated with the medical procedure. In some embodiments, the trained machine learning model may be used to identify and/or determine location information of one or more objects of interest in the inputted specific image data that cause the anomaly regarding the specific medical procedure in response to the determination that the anomaly regarding the specific medical procedure exists.

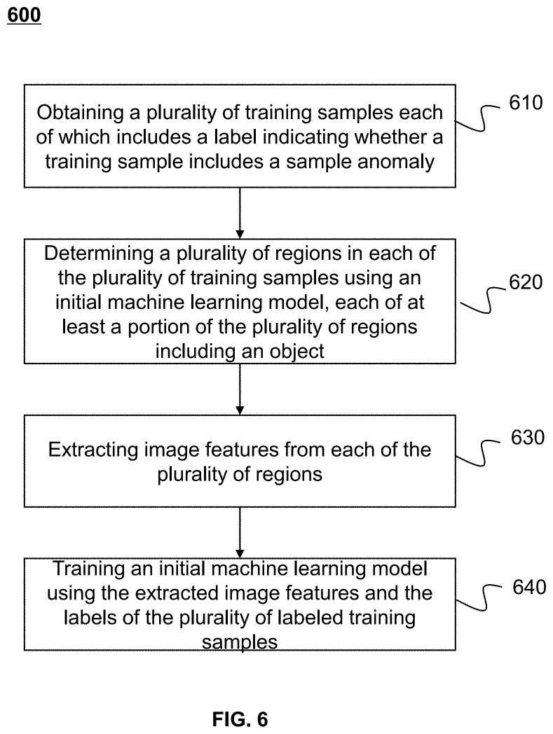

In some embodiments, the processing device 130 may retrieve the trained machine learning model from the storage device 150 or any other storage device. For example, the trained machine learning model may be obtained by training a machine learning model offline using a processing device different from or same as the processing device 130. The processing device may store in the trained machine learning model in the storage device 150 or any other storage device The processing device 130 may retrieve the trained machine learning model from the storage device 150 or any other storage device in response to receipt of a request for anomaly detection. More descriptions regarding the training of the machine learning model for anomaly detection may be found elsewhere in the present disclosure. See, e.g., FIG. 6, and relevant descriptions thereof.

In 530, the processing device 130 (e.g., the determination module 420) may determine a detection result for the medical procedure using the trained machine learning model based on the image data.

In some embodiments, the detection result for the medical procedure may indicate whether the anomaly regarding the medical procedure exists. In some embodiments, an object which may cause damage or abnormity to the medical device (e.g., the medical device 110), the individual (e.g., a doctor, the patient, etc.), etc., may be also referred to as an anomaly. For example, the anomaly in an MR or X-ray scan may include one or more magnetically active elements (e.g., a metal accessory of a patient (e.g., a watch, jewelry, a hair pin), a wheelchair for the patient, etc.) present in an MR room during an MR scan, which may cause serious threat to the patient and/or damage to the MR scanner. As another example, the anomaly in a surgery procedure may include one or more alien objects (e.g., a sponge) inadvertently left within a patient's body after the surgery, which may cause harm to the patient. As a further example, the anomaly in a medical procedure may include one or more objects which are not at positions predetermined according to a criterion, such as a scanning protocol, an operative regulation, etc. As still another example, the anomaly in a medical procedure may include an obstacle in the trajectory of a medical device moving in the medical procedure. In some embodiments, an anomaly in a medical procedure may include an event that may cause damage or abnormity to the medical device (e.g., the medical device 110), an individual (e.g., a doctor, the patient, etc.), etc., associated with the medical procedure. For example, an anomaly in a medical procedure may include an abnormal setting of a medical device (e.g., the location of a scanning table) involved in the medical procedure. As another example, an anomaly in a medical procedure may include abnormal behaviors of individuals (e.g., a patient) in the medical procedure. As a further example, an abnormal behavior of an individual may include that the patient is improperly positioned, that an individual moves toward or is located at a dangerous location, etc.

In some embodiments, the detection result for the medical procedure may include a positive result or a negative result. The positive result may indicate the existence of the anomaly regarding the medical procedure. The negative result may indicate the inexistence of the anomaly in the image data. The processing device 130 may input the image data into the trained machine learning model. The processing device 130 may generate the detection result based on the inputted image data. For example, the trained machine learning model may divide the image data (e.g., an image or a video) into one or more regions (or segments, or instances). The processing device 130 may determine a predicted result for each of the one or more regions (or segments, or instances). The predicted result for a region (or a segment, or an instance) may indicate whether the region (or a segment, or an instance) includes an object of interest that causes the anomaly of the image data. In other words, the predicted result for a region (or a segment, or an instance) may indicate whether the anomaly regarding the medical procedure exists in the region. The predicted result for a region (or a segment, or an instance) may include a predicted positive result or a predicted negative result. The predicted positive result for a region may indicate the region (or a segment, or an instance) includes an object of interest that causes the anomaly regarding the medical procedure. The predicted negative result for a region may indicate the region (or a segment, or an instance) includes an object of uninterest that does not cause the anomaly regarding the medical procedure or lacks an object of interest. In some embodiments, the predicted positive result may be denoted by a positive label, such as “1.” The predicted negative result may be denoted by a negative label, such as “0.” The processing device 130 may determine the detection result for the image data based on predicted results of the one or more regions. For example, if all the predicted results for the one or more regions are negative, i.e., the predict labels for the one or more regions are negative label, the processing device 130 may determine that the anomaly regarding the medical procedure does not exist. The detection result regarding the medical result may be a negative result. If at least one of the predicted results for the one or more regions is positive, i.e., at least one of the predict labels for the one or more regions is a positive label, the processing device 130 may determine that the anomaly regarding the medical procedure exists. The detection result regarding the medical procedure may be a positive result.

In some embodiments, the inputted image data (i.e., an image, a video) may be divided into multiple segments or regions each of which includes representation of an object. Image features may be extracted from each segment or instance. The one or more image features extracted and/or output by the object detection model may be also referred to as a feature map or vector. Exemplary image features may include a low-level feature (e.g., an edge feature, a textural feature), a high-level feature (e.g., a semantic feature), a complicated feature (e.g., a deep hierarchical feature), etc. The processing device 130 may determine the predicted result for a specific region based on image features extracted from the specific region in the image data. For example, based on the extracted image features, the trained machine learning model may determine an anomaly score for the specific region and determine the predicted result based on the anomaly score for the specific region. For example, if the trained machine learning model determines that the anomaly score of the specific region is greater than an anomaly threshold, the trained machine learning model may determine that the detection result of the specific region is positive and/or designate a positive label “1” for the specific region; otherwise, the trained machine learning model may determine that the predicted result of the specific region is negative and/or designate a negative label “0” for the specific region.

In some embodiments, the trained machine learning model may determine an anomaly score based on extracted image features of the multiple segments or regions. An anomaly score may indicate a probability that the inputted image data includes the anomaly. The trained machine learning model may determine whether the anomaly regarding the medical procedure exists based on the anomaly score. For example, the trained machine learning model may compare the anomaly score with an anomaly threshold, and if the anomaly score exceeds the anomaly threshold, the trained machine learning model may determine that the anomaly regarding the medical procedure exists. In some embodiments, the trained machine learning model may determine an anomaly score based on the extracted image features from each of the multiple segments or regions. Each of the multiple segments or regions may be assigned an anomaly score. The trained machine learning model may determine whether the anomaly regarding the medical procedure exists based on one or more of anomaly scores corresponding to the multiple segments or regions. For example, the trained machine learning model may compare a maximum score among the anomaly scores with an anomaly threshold, and if the maximum score exceeds the anomaly threshold, the trained machine learning model may determine that the anomaly regarding the medical procedure exists. An anomaly score for a specific region may be determined based on a probability generation function of the trained machine learning. The probability generation function of the trained machine learning may include a logistic function, a sigmoid function, etc.

In some embodiments, in response to a determination that the image data includes the anomaly, the trained machine learning model may be used to identify and/or determine location information of one or more objects of interest in the image data that cause the anomaly regarding the medical procedure in response to the determination that the anomaly regarding the medical procedure exists based on the image data. In other words, the trained machine learning model may classify one or more objects present in the inputted image data into two categories including a positive category and a negative category. An object belonging to the negative category (also referred to as an object of uninterest) may not cause the anomaly regarding the specific medical procedure. An object belonging to the positive category (also referred to as an object of interest) may cause the anomaly regarding the medical procedure. In some embodiments, the trained machine learning model may be configured to mark and/or locate an object of interest that causes the anomaly regarding the medical procedure in the inputted image data using a bounding box. The bounding box may refer to a box enclosing at least a portion of the detected object of interest in the image data. The bounding box may be of any shape and/or size. For example, the bounding box may have the shape of a square, a rectangle, a triangle, a polygon, a circle, an ellipse, an irregular shape, or the like. In some embodiments, the bounding box may be a minimum bounding box that has a preset shape (e.g., a rectangle, a square, a polygon, a circle, an ellipse) and completely encloses a detected object of interest. As used herein, a minimum bounding box that has a preset shape (e.g., a rectangle, a square, a polygon, a circle, an ellipse) and completely encloses a detected object of interest indicates that if a dimension of the minimum bounding box (e.g., the radius of a circle minimum bounding box, the length or width of a rectangular minimum bounding box, etc.) is reduced, at least a portion of the detected object of interest is outside the minimum bounding box. The trained machine learning model may be configured to output at least a portion of the processed image data with a bounding box that marks a detected object of interest. For instance, the trained machine learning model may be configured to output the bounding box with the detected object of interest that causes the anomaly regarding the medical procedure.

In some embodiments, the trained machine learning model may be configured to track an object of interest in the inputted image data (e.g., two adjacent frames of a video). For example, the trained machine learning model may determine a similarity degree of two objects of interest present in two adjacent frames of the inputted image data (e.g., a video). If the similarity degree of two objects of interest present in the two adjacent frames of the video satisfies a condition, the trained machine learning model may designate the two objects of interest as one same object of interest.

In 540, the processing device (e.g., the feedback module 430) may provide feedback relating to the anomaly in response to the detection result that the anomaly exists.

In some embodiments, the feedback provided by the processing device 130 may include the detection result that the anomaly regarding the medical procedure exists. For example, the processing device 130 may generate a notification for notifying that the anomaly exists. The notification for notifying that the anomaly exists may be transmitted to a device (e.g., the terminal(s) 140). The device (e.g., the terminal(s) 140) may be caused to play and/or display the notification to related personal (e.g., a patient, a doctor) for notifying that the anomaly exists. The feedback or notification relating to the anomaly regarding the medical procedure may be in the form of image, text, voice, etc. For example, before an MR scan is performed on a patient, a wheelchair may be left in a scanning room. The terminal(s) 140 may receive the notification and sets off an alarm to notify an operator of the MR scan that the anomaly regarding the MR scan exists. As another example, the terminal(s) 140 may display the notification as text such as “Alien Object!” to notify an operator of the MR scan that the anomaly regarding the MR scan exists.

In some embodiments, the detection result may include the location information of at least one of the one or more objects that cause the anomaly regarding the medical device. The feedback or notification provided by the processing device 130 may include the location information of the at least one of the one or more objects that cause the anomaly regarding the medical device. For example, the processing device 130 may transmit at least a portion of the image data with the detected and/or marked object(s) of interest that cause the anomaly regarding the medical procedure to a device (e.g., the terminal(s) 140). The device may be caused to present at least a portion of the received image data. The device may also present the location information of the at least one of objects of interests. The location information of the at least one of objects of interests may be part of the received image data. For example, the location information of the at least one of objects of interests may be denoted as a bounding box as described above. In some embodiments, the device may highlight at least one of the one or more objects of interest in the presentation. For example, the device may highlight an area covered by a bounding box enclosing an object of interest using a color different from other areas surrounding the object of interest.

It should be noted that the above description is merely provided for the purpose of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the processing device may preprocess the image data after the processing device 130 obtaining the image data. The preprocessing of the image data may include cropping, taking a snapshot, scaling, denoising, rotating, recoloring, subsampling, background elimination, normalization, or the like, or any combination thereof. In some embodiments, the processing device 130 may obtain audio data acquired by one or more sound detectors. The audio data may be coupled with the image data. In some embodiments, the audio data may be converted into text data such as one or more sentence, words, a paragraph, etc., using a voice recognition technique. The trained machine learning model may determine whether the anomaly regarding the medical procedure exists based on the text data and/or the one or more images in the image data. In some embodiments, the audio data may be inputted into the trained machine learning model together with the image data. The trained machine learning model may determine whether the anomaly regarding the medical procedure exists based on the audio data and/or the image data. In some embodiments, operation 540 may be omitted.

FIG. 6 is a flowchart illustrating an exemplary process 600 of training a machine learning model according to some embodiments of the present disclosure. In some embodiments, process 600 may be an offline process. The process 600 may be executed by the anomaly detection system 100. For example, the process 600 may be implemented as a set of instructions (e.g., an application) stored in a storage device in the processing device 130. The processing device 130 may execute the set of instructions and accordingly be directed to perform the process 600 in the anomaly detection system 100. The operations of the illustrated process 600 presented below are intended to be illustrative. In some embodiments, the process 600 may be accomplished with one or more additional operations not described, and/or without one or more of the operations discussed. Additionally, the order in which the operations of the process 600 as illustrated in FIG. 6 and described below is not intended to be limiting.

In 610, the processing device 130 (e.g., the obtaining module 450) may obtain a plurality of training samples. In some embodiments, the plurality of training samples may include negative samples. If a training sample includes image data having no anomaly regarding a medical procedure associated with the training sample, the training sample may be a negative sample or normal sample. In some embodiments, the plurality of training samples may include positive samples. If a training sample includes image data having an anomaly like a patient wearing a watch in an MR scanning room, the training sample may be labeled as a positive sample or abnormal sample. Each of the plurality of training samples may include historical image data collected by one or more visual sensors via monitoring a historical medical procedure in a historical time period (e.g., the past one or more years, the past one or more months). For example, a training sample may include one or more static images captured by the one or more visual sensors. In some embodiments, the training samples may be obtained from the monitoring device 120 or acquired from a storage device (e.g., the storage device 150, an external data source), the terminal(s) 140, or any other storage device.

The sample anomaly (i.e., anomaly) regarding a medical procedure associated with the training sample may refer to that the training sample includes one or more objects of interest, which may cause damage or abnormity to the medical device (e.g., the medical device 110), an individual (e.g., a doctor, the patient, etc.), etc., associated with the medical procedure as described elsewhere in the present disclosure (e.g., FIG. 5 and the descriptions thereof). In some embodiments, the plurality of training samples may be all negative training samples (or negative samples). All objects presented in a negative training sample may not cause the anomaly regarding a medical procedure associated with the negative training sample. Using the negative training samples, a machine learning model may be trained to learn what normal conditions or scenarios may be like and therefore configured to detect deviations from normal conditions or scene in order to identify anomaly. In some embodiments, the plurality of training samples may include a first portion and a second portion. The first portion may include a plurality of negative training samples. The second portion may include a plurality of positive training samples (or positive samples). A ratio of a count or number of the plurality of negative training samples in the first portion to a count or number of the plurality of positive training samples in the second portion may be a constant. The constant may be a default setting of the anomaly detection system 100. The greater the ratio of the count or number of the plurality of negative training samples to the count or number of the plurality of positive training samples in the plurality of training samples, the higher the detection rate of a trained machine learning model generated based on the plurality of training samples may be, and the higher the false positive rate of the trained machine learning model may be. The detection rate of the trained machine learning model may be also referred to as a sensitivity degree of the trained machine learning model. The detection rate of the trained machine learning model may be increased by increasing the proportion of the positive training samples among the plurality of training samples. The false-positive rate may be decreased by increasing the proportion of the negative training samples among the plurality of training samples. It may be desired that the trained machine learning model provides a high detection rate and a low false-positive rate. In order to reach a desired balance between the two performance criteria including the detection rate and false-positive rate, the ratio of the count of the plurality of positive training samples to the count of the plurality of negative training samples may be close or equal to the actual occurrence rate of anomaly in clinical applications. For example, the actual occurrence rate of anomaly in the clinical application may be determined based on historical medical procedures in a historical time period (e.g., past one year). Further, the number or count of historical medical procedures including anomaly and the number or count of historical medical procedures having no anomaly in the historical time period may be statistically determined. The ratio of the count of the plurality of positive training samples to the count of the plurality of negative training samples may be close or equal to a ratio of the number or count of historical medical procedures including anomaly to the number or count of historical medical procedures having no anomaly in the historical time period.

In some embodiments, a trained machine learning model (e.g., the trained machine learning model determined in operation 640) may be provided by training an initial machine learning model using the plurality of training samples based on a weakly supervised learning technique. Exemplary weakly supervised learning techniques may include an incomplete supervised learning technique (e.g., an active learning technique and a semi-supervised learning technique), an inexact supervised learning technique (e.g., a multi-instance learning technique), an inaccurate supervised learning technique, etc. Using the weakly supervised learning technique, each of the plurality of training samples may be tagged with a label indicating whether each of the plurality of training samples includes an anomaly regarding a historical medical procedure. If a training sample includes an anomaly regarding a historical medical procedure, the training sample may be a positive training sample. If a training sample does not include an anomaly regarding a historical medical procedure, the training sample may be a negative training sample. The training label of a sample may be at the image-level or video level. In other words, the training label (anomalous or normal) of a training sample may be tagged or known, while training labels (anomalous or normal) of one or more objects presented in the training sample may be unknown or untagged. The label of a training sample may include a positive label or a negative label. A training sample may be tagged with a negative label if the training sample is a negative training sample with no sample anomaly. A training sample may be tagged with a positive label if the training sample is a positive training sample with sample anomaly. A training sample may be tagged with a binary label (e.g., 0 or 1, positive or negative, etc.). For example, a negative training sample may be tagged with a negative label (e.g., “0”), while a positive training sample may be tagged with a positive label (e.g., 1).

In 620, the processing device 130 (e.g., the extraction module 460) may determine a plurality of regions in each of the plurality of training samples using an initial machine learning model. In some embodiments, the initial machine learning model may include a machine learning model that has not been trained using any training data. For example, the initial machine learning model may include structural parameters, such as a count or number of layers, a count or number of nodes for each layer, etc., and learning parameters, such as connected weights, bias vectors, etc. The structural parameters of the initial machine learning model may be set by an operator of the processing device 130 which may be not updated in a training process of the initial machine learning model. The learning parameters may be unknown as the initial machine learning model has not been trained using any training data and be updated in the training process of the initial machine learning model using the plurality of training samples obtained in 610. In some embodiments, the initial machine learning model may include a pre-trained machine learning model that may be trained using a training set. Training data in the training set may be partially or entirely different from the plurality of training samples obtained in 610. For example, the pre-trained machine learning model may be provided by a system of a vendor who provides and/or maintains such a pre-trained machine learning model. The structural parameters of the initial machine learning model may be set by the vendor who provides and/or maintains such a pre-trained machine learning model. The learning parameters of the pre-trained machine learning model may be pre-determined using the training set and may further be updated based on the plurality of training samples obtained in 610.

In some embodiments, the initial machine learning model may be constructed based on a neural network model, a deep learning model, a regression model, etc. Exemplary neural network models may include an artificial neural network (ANN), a convolutional neural network (CNN) (e.g., a region-based convolutional network (R-CNN), a fast region-based convolutional network (Fast R-CNN), a faster region-based convolutional network (Faster R-CNN), etc.), a spatial pyramid pooling network (SPP-Net), etc., or the like, or any combination thereof. Exemplary deep learning models may include one or more deep neural networks (DNN), one or more deep Boltzmann machines (DBM), one or more stacked autoencoders, one or more deep stacking networks (DSN), etc. Exemplary regression models may include a support vector machine, a logical regression model, etc.

In some embodiments, the initial machine learning model may include a multi-layer structure. For example, the initial machine learning model may include an input layer, an output layer, and one or more hidden layers between the input layer and the output layer. In some embodiments, the hidden layers may include one or more convolution layers, one or more rectified-linear unit layers (ReLU layers), one or more pooling layers, one or more fully connected layers, or the like, or any combination thereof. As used herein, a layer of a model may refer to an algorithm or a function for processing input data of the layer. Different layers may perform different kinds of processing on their respective input. A successive layer may use output data from a previous layer of the successive layer as input data. In some embodiments, the convolutional layer may include a plurality of kernels, which may be used to extract a feature of the image data. In some embodiments, each kernel of the plurality of kernels may filter a portion (i.e., a region) of the image data to extract a specific image feature corresponding to the portion. The specific image feature may be determined based on the kernels. Exemplary image features may include a low-level feature (e.g., an edge feature, a textural feature), a high-level feature, or a complicated feature. The pooling layer may take an output of the convolutional layer as an input. The pooling layer may include a plurality of pooling nodes, which may be used to sample the output of the convolutional layer, so as to reduce the computational load of data processing and accelerate the speed of data processing speed. In some embodiments, the size of the matrix representing the image data may be reduced in the pooling layer. The fully connected layer may include a plurality of neurons. The neurons may be connected to the pooling nodes in the pooling layer. In the fully connected layer, a plurality of vectors corresponding to the plurality of pooling nodes may be determined based on one or more image features of a training sample, and a plurality of weighting coefficients may be assigned to the plurality of vectors. The output layer may determine an output based on the vectors and the weighting coefficients obtained from the fully connected layer. In some embodiments, an output of the output layer may include a probability map, a classification map, and/or a regression map.

In some embodiments, each of the layers may include one or more nodes. In some embodiments, each node may be connected to one or more nodes in a previous layer. The number of nodes in each layer may be the same or different. In some embodiments, each node may correspond to an activation function. As used herein, an activation function of a node may define an output of the node given an input or a set of inputs. In some embodiments, each connection between two of the plurality of nodes in the initial machine learning model may transmit a signal from one node to another node. In some embodiments, each connection may correspond to a weight. As used herein, a weight corresponding to a connection may be used to increase or decrease the strength or impact of the signal at the connection.

In some embodiments, the plurality of regions may be determined using the initial machine learning model based on a sliding window algorithm, a region proposal algorithm, an image segmentation algorithm, etc. For example, using the sliding window algorithm, the initial machine learning model may divide the image data into a plurality of regions by sliding a window with a fixed size. As another example, using the region proposal algorithm, the initial machine learning model may be configured to designate each pixel in an inputted training sample as a group. The initial machine learning model may be configured to determine a texture feature of each group and determine a similarity degree between two groups. The initial machine learning model may combine multiple groups each two of which includes the similarity degree satisfies a condition, such as exceeding a threshold. In some embodiments, the initial machine learning model may extract a preliminary outline or contour of each of one or more objects to be recognized in a training sample using an image segmentation algorithm (e.g., an edge detection algorithm). The processing device 130 may divide a region covering the preliminary outline or contour of each of the one or more objects. In some embodiments, a region may be determined based on one or more feature points (e.g., an inflection point, a boundary or edge point of an object) in the image data. As used herein, a feature point may refer to a point where the gray value of an image changes dramatically or where the curvature of an edge is larger (i.e., the intersection of two edges). Specifically, after one or more specific feature points are identified in the image data, a region of a predetermined shape or size may be determined within which the specific feature points are located. In some embodiments, a shape of each of the plurality of regions may include a rectangle, a circle, an ellipse, a polygon, an irregular shape, etc. In some embodiments, at least one parameter of the size, shape, or count of the plurality of regions may be assigned default values determined by the anomaly detection system 100 or preset by a user or operator via the terminal(s) 140. In some embodiments, each of one or more parameters may be assigned a value, while one or more parameters may be determined based on the assigned values. For instance, the size and shape of each of the plurality of regions may be assigned, while the count of the plurality of regions may be determined based on the size and shape of each of the plurality of region.

In 630, the processing device 130 (e.g., the extraction module 460) may extract image features from each of the plurality of regions.

An image feature may refer to a representation of a specific structure in a region of a training sample, such as a point, an edge, an object, etc. The extracted image features may be binary, numerical, categorical, ordinal, binomial, interval, text-based, or combinations thereof. In some embodiments, an image feature may include a low-level feature (e.g., an edge feature, a textural feature), a high-level feature (e.g., a semantic feature), or a complicated feature (e.g., a deep hierarchical feature). The initial machine learning model may process the inputted training sample via multiple layers of feature extraction (e.g., convolution layers) to extract image features.

In 640, the processing device 130 (e.g., the training module 470) may train the initial machine learning model using the extracted image features and the plurality of labeled training samples.

In some embodiments, the trained machine learning model may be obtained by training the initial machine learning model based on the extracted image features of each of the plurality of training samples using a training algorithm. Exemplary training algorithms may include a gradient descent algorithm, a Newton's algorithm, a Quasi-Newton algorithm, a Levenberg-Marquardt algorithm, a conjugate gradient algorithm, or the like, or a combination thereof. In some embodiments, the initial machine learning model may be trained by performing a plurality of iterations. Before the plurality of iterations, the parameters of the initial machine learning model may be initialized. For example, the connected weights and/or the bias vector of nodes of the initial machine learning model may be initialized by assigning random values in a range, e.g., the range from −1 to 1. As another example, all the connected weights of the initial machine learning model may be assigned a same value in the range from −1 to 1, for example, 0. As still an example, the bias vector of nodes in the initial machine learning model may be initialized by assigning random values in a range from 0 to 1. In some embodiments, the parameters of the initial machine learning model may be initialized based on a Gaussian random algorithm, a Xavier algorithm, etc. Then the plurality of iterations may be performed to update the parameters of the initial machine learning model until a termination condition is satisfied. The termination condition may provide an indication of whether the initial machine learning model is sufficiently trained. For example, the termination condition may be satisfied if the value of a cost function or an error function associated with the initial machine learning model is minimal or smaller than a threshold (e.g., a constant). As another example, the termination condition may be satisfied if the value of the cost function or the error function converges. The convergence may be deemed to have occurred if the variation of the values of the cost function or the error function in two or more consecutive iterations is smaller than a threshold (e.g., a constant). As still an example, the termination condition may be satisfied when a specified number or count of iterations are performed in the training process. For each of the plurality of iterations, image features of each of the plurality of regions of a training sample and the corresponding label may be inputted into the initial machine learning model. The image features may be processed by one or more layers of the initial machine learning model to generate a predicted result for each region of the plurality of regions in the inputted training sample. The predicted result for a specific region may indicate whether the specific region includes the sample anomaly. In other words, the predicted result for the specific region may indicate whether the specific region includes an object of interest that causes the anomaly of the training sample. In some embodiments, the predicted result for the specific region may include a positive result indicating that the specific region includes the anomaly or a negative result indicating that the specific region has no anomaly. The initial machine learning model may determine the predicted result for the specific region by determining an anomaly score for the specific region based on the image features extracted from the specific region. For example, if the anomaly score for the specific region exceeds an anomaly threshold, the initial machine learning model may determine that the predicted result for the specific region is positive. For instance, the positive result may be denoted by value “1.” If the anomaly score for the specific region is less than the anomaly threshold, the initial machine learning model may determine that the predicted result for the specific region is negative. For instance, the negative result may be denoted by value “0.” In some embodiments, the predicted result for the specific region may include the anomaly score for the specific region. Each of predicted results of the plurality of regions in the inputted training sample may be compared with a desired result (i.e., the label) associated with the training sample based on the cost function or error function of the initial machine learning model. The cost function or error function of the initial machine learning model may be configured to assess a total difference (also referred to as a global error) between testing values (e.g., the predicted results of each of the regions) of the initial machine learning model and a desired value (e.g., the label of the training sample). The total difference (also referred to as a global error) between testing values (e.g., the predicted results of each of the regions) of the initial machine learning model and a desired value (e.g., the label of the training sample) may be equal to a sum of multiple differences each of which is between one of the predicted results of the plurality of regions and the label of the inputted training sample. If the value of the cost function or error function exceeds a threshold in a current iteration, the parameters of the initial machine learning model may be adjusted and/or updated to cause the value of the cost function or error function to reduce to a value smaller than the threshold. Accordingly, in a next iteration, image features of each region in another training sample may be inputted into the initial machine learning model to train the initial machine learning model as described above until the termination condition is satisfied.

In some embodiments, the termination condition may be that a value of a cost function or error function in the current iteration is less than a threshold value. In some embodiments, the termination conditions may include that a maximum number (or count) of iterations has been performed, that an approximation error is less than a certain threshold, a difference between the values of the cost function or error function obtained in a previous iteration and the current iteration (or among the values of the cost function or error function within a certain number or count of successive iterations) is less than a certain threshold, that a difference between the approximation error at the previous iteration and the current iteration (or among the approximation errors within a certain number or count of successive iterations) is less than a certain threshold. In response to a determination that the termination condition is not satisfied, the processing device 130 may adjust the parameters of the initial machine learning model, and perform the iterations. For example, the processing device 130 may update values of the parameters by performing a backpropagation machine learning training algorithm, e.g., a stochastic gradient descent backpropagation training algorithm. In response to a determination that the termination condition is satisfied, the iterative process may terminate and the trained machine learning model may be stored and/or output. In some embodiments, after learning is complete, a validation set may be processed to validate the results of learning. In some embodiments, the trained machine learning model may be stored in a storage device (e.g., the storage device 150), the processing device 130, the terminal(s) 140, or an external data source. The processing device 130 may obtain the trained machine learning model to perform anomaly detection.

In some embodiments, the trained machine learning model may include two components. One of the two components may be trained to detect whether the anomaly regarding the medical procedure exists, which may be also referred to as an anomaly detection component. Another one of the two components may be trained to determine and/or output the locations of the one or more objects of interest that cause the anomaly, which may be also referred to as a classification component. The two components may be connected with each other. In some embodiments, the output of the anomaly detection component may be an input of the classification component. The classification component may determine one or more objects of interest that cause the anomaly detected by the anomaly detection component. In some embodiments, the two components may share the same multiple layers for extracting image features from inputted image data. The extracted image features may be inputted into each of the two components, respectively. Each of the two components may generate an output based on the extracted image features.

It should be noted that the above description is merely provided for the purposes of illustration, and not intended to limit the scope of the present disclosure. For persons having ordinary skills in the art, multiple variations and modifications may be made under the teachings of the present disclosure. For example, the operation 620 of determining the plurality of regions and the operation 630 of extracting the image features may be integrated into the operation 640 of training the initial to determine the plurality of regions and image features for training the initial machine model. As another example, an update process of the trained machine learning model may be added to update the trained machine learning model periodically or at multiple different times. However, those variations and modifications do not depart from the scope of the present disclosure.

### EXAMPLES

The following examples are provided for illustration purposes and are not intended to limit the scope of the present disclosure.

### Example 1 Exemplary Detection Result of a Surgery Procedure

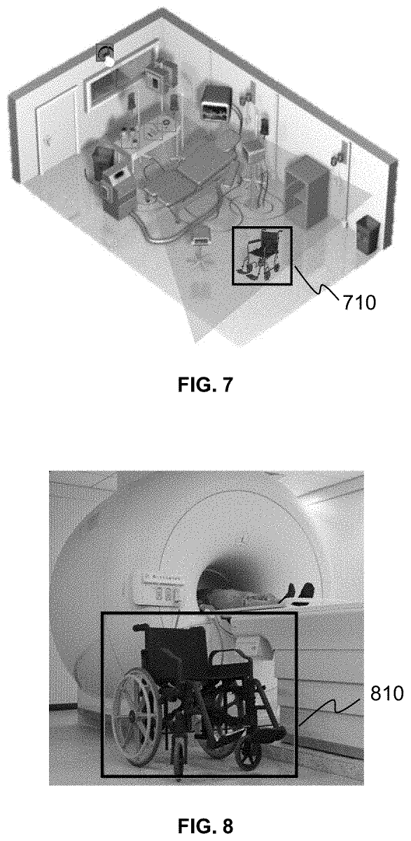

FIG. 7 is a schematic diagram illustrating a detection result regarding an exemplary medical procedure according to some embodiments of the present disclosure. As shown in FIG. 7, a wheelchair was detected by a trained machine learning model and marked using a bounding box 710 in an image depicting the surgery procedure. The wheelchair was located on the trajectory of a medical device moving in the surgery procedure, which was determined to cause the anomaly in the surgery procedure.

### Example 2 Exemplary Detection Result of an Imaging Scan

FIG. 8 is a schematic diagram illustrating a detection result regarding another exemplary medical procedure according to some embodiments of the present disclosure. As shown in FIG. 8, a wheelchair was detected by a trained machine learning model and marked using a bounding box 810 in an image associated with the imaging scan. The wheelchair may cause damage or abnormity to a medical device (e.g., an MR scanner) used for performing the imaging scan, which was determined to cause the anomaly in the imaging scan.

### Example 3 Exemplary Detection Result of a Surgery Procedure

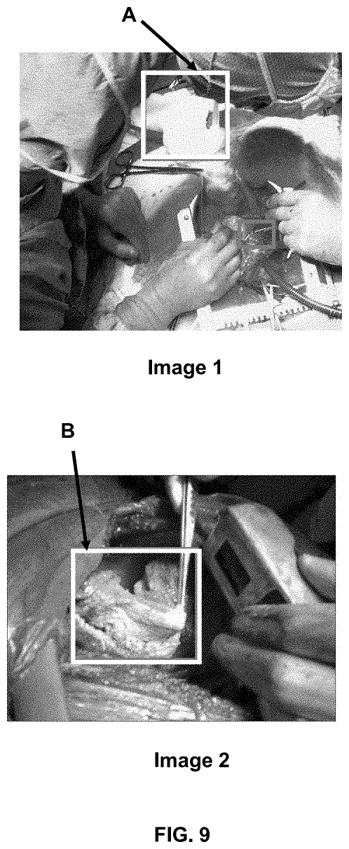

FIG. 9 is a schematic diagram illustrating an anomaly detection of an exemplary surgery procedure according to some embodiments of the present disclosure. As shown in FIG. 9, Image 1 and Image 2 were collected by a camera during a surgery procedure. In some embodiments, Image 1 and Image 2 may be two frames in a video collected by the camera. Each of the Image 1 and Image 2 had a timestamp indicating a time point when each of the Image 1 and Image 2 was collected. The timestamps reveal that Image 2 was acquired later than Image 1. A sponge used in the surgery procedure was detected in Image 1 using a trained machine learning model and marked using a bounding box A according to process 500 as described elsewhere in the present disclosure. The sponge may cause damage or abnormity to a patient on whom the surgery procedure was performed if the sponge was inadvertently left behind in a patient's body after the surgery. The sponge detected in Image 1 was tracked in the surgery procedure continuously. For example, the sponge used in the surgery procedure was detected in Image 2 and marked using a bounding box B. Images with the marked sponge (e.g., Image 1 and Image 2) generated in the surgery procedure may be displayed to a surgeon on a device (e.g., a terminal device), and thus the surgeon may know the locations of the sponge at various time points in the surgery procedure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer-readable media having computer-readable program code embodied thereon.

A non-transitory computer-readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer-readable signal medium may be any computer-readable medium that is not a computer-readable storage medium and that may communicate, propagate, or transport a program for use by or in connection with an instruction execution system, apparatus, or device. Program code embodied on a computer-readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object-oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby, and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

We claim:

1. A system for anomaly detection for a medical procedure, comprising:
at least one storage device storing executable instructions, and
at least one processor in communication with the at least one storage device, when executing the executable instructions, causing the system to perform operations including:
obtaining image data collected by one or more visual sensors via monitoring a medical procedure;
obtaining a trained machine learning model for anomaly detection;
determining, based on the image data, a detection result for the medical procedure using the trained machine learning model, the detection result including whether an anomaly regarding the medical procedure exists; and
in response to the detection result that the anomaly exists, providing feedback relating to the anomaly.

2. The system of claim 1, wherein to provide feedback relating to the anomaly, the at least one processor is further configured to cause the system to perform additional operations including:
generating a notification for notifying that the anomaly exists.

3. The system of claim 1, wherein the image data include representation of the one or more objects of interest that cause the anomaly.

4. The system of claim 3, wherein the detection result for the medical procedure includes location information of at least one of the one or more objects of interest.

5. The system of claim 4, wherein to determine, based on the image data, a detection result for the medical procedure using the trained machine learning model, the at least one processor is further configured to cause the system to perform additional operations including:
in response to the detection result that the anomaly regarding the medical procedure exists, determining, based on the image data, the location information of at least one of the one or more objects of interest using the trained machine learning model.

6. The system of claim 5, wherein to determine location information of at least one of one or more objects of interest, the at least one processor is further configured to cause the system to perform additional operations including:
extracting a plurality of regions represented in the image data;
determining a score of each of the plurality of regions, the score of each of the plurality of regions denoting a probability that the each of the plurality of regions includes the at least one of the one or more objects of interest; and
determining, based on the score of each of the plurality of regions, the location information of the at least one of the one or more objects of interest in the image data.

7. The system of claim 3, wherein to provide feedback relating to the anomaly, the at least one processor is further configured to cause the system to perform additional operations including:
causing at least a portion of the image data to be presented as a presentation on a device; and
causing the at least one of the one or more objects to be highlighted in the presentation.

8. The system of claim 7, wherein the presentation is in a form of a video or a static image.

9. The system of claim 1, wherein the trained machine learning model for anomaly detection is constructed based on a weakly supervised learning model.

10. The system of claim 1, wherein the trained machine learning model is provided by operations including:
obtaining a plurality of training samples each of which includes a label indicating whether a training sample includes a sample anomaly;
determining a plurality of regions in each of the plurality of training samples, each of at least a portion of the plurality of regions including an object;
extracting image features from each of the plurality of regions; and
training an initial machine learning model using the extracted image features and the labels of the plurality of training samples.

11. The system of claim 10, wherein the plurality of training samples include a plurality of negative training samples each of which has no sample anomaly.

12. The system of claim 10, wherein the plurality of training samples include a first portion and a second portion, the first portion includes a plurality of negative training samples each of which has no sample anomaly, and the second portion includes a plurality of positive training samples each of which includes a sample anomaly.

13. The system of claim 1, wherein the trained machine learning model is constructed based on a neural network model.

14. A method implemented on a computing device having at least one processor and at least one storage device for anomaly detection for a medical procedure, the method comprising:
obtaining image data collected by one or more visual sensors via monitoring a medical procedure;
obtaining a trained machine learning model for anomaly detection;
determining, based on the image data, a detection result for the medical procedure using the trained machine learning model, the detection result including whether an anomaly regarding the medical procedure exists; and
in response to the detection result that the anomaly exists, providing feedback relating to the anomaly.

15. The method of claim 14, wherein to provide feedback relating to the anomaly, the method includes:
causing at least a portion of the image data to be presented as a presentation on a device; and
causing the at least one of the one or more objects to be highlighted in the presentation.

16. The method of claim 14, wherein the trained machine learning model for anomaly detection is constructed based on a weakly supervised learning model.

17. The method of claim 14, wherein the trained machine learning model is provided by operations including:
obtaining a plurality of training samples each of which includes a label indicating whether a training sample includes a sample anomaly; and
training an initial machine learning model using the plurality of training samples.

18. The method of claim 17, wherein the plurality of training samples include a plurality of negative training samples each of which has no sample anomaly.

19. The method of claim 17, wherein the plurality of training samples include a first portion and a second portion, the first portion includes a plurality of negative training samples each of which has no sample anomaly, and the second portion includes a plurality of positive training samples each of which includes a sample anomaly.

20. A non-transitory computer readable medium, comprising a set of instructions for anomaly detection for a medical procedure, wherein when executed by at least one processor, the set of instructions direct the at least one processor to effectuate a method, the method comprising:
obtaining image data collected by one or more visual sensors via monitoring a medical procedure;
obtaining a trained machine learning model for anomaly detection;
determining, based on the image data, a detection result for the medical procedure using the trained machine learning model, the detection result including whether an anomaly regarding the medical procedure exists; and
in response to the detection result that the anomaly exists, providing feedback relating to the anomaly.
